(19) United States
(12) Patent Application Publication
(10) Pub. No.: US 2022/0223291 A1
(43) Pub. Date: Jul. 14, 2022

(54) Platform for Facilitating the Analysis of Medical Survey Data and Connecting Medical Practitioners with Patients

(71) Applicant: MDDirect Ltd., St. Paul, MN (US)

(72) Inventors: Samuel Moen, Hudson, WI (US); Jack Bert, Woodbury, MN (US)

(21) Appl. No.: 17/569,842

(22) Filed: Jan. 6, 2022

Related U.S. Application Data
(60) Provisional application No. 63/135,543, filed on Jan. 8, 2021.

(51) Int. Cl. G16H 50/30; G16H 10/60; G16H 50/70; G16H 15/00

(52) U.S. Cl. CPC G16H 50/30; G16H 15/00; G16H 50/70; G16H 10/60

(57) ABSTRACT

This specification generally describes technology for facilitating the analysis of medical survey data and connecting medical practitioners with patients. In some implementations, a system includes a medical condition data model that provides associations between medical assessment data and areas of concern, a medical practitioner data repository that provides a mapping between medical symptoms and medical practitioners, and one or more computing servers, patient computing devices, and medical practitioner computing devices. Medical survey data is provided to a patient computing device, and responses to survey questions are received from the patient computing device. The medical condition data model is used to analyze the responses, and annotate the medical survey data to indicate areas of concern. The medical practitioner data repository is accessed to select medical practitioners that specialize in recommending treatment based on the responses, and a recommendation for treatment of the patient is received from a medical practitioner computing device.

### CROSS-REFERENCE TO RELATED APPLICATIONS

This application claims the benefit of U.S. Provisional Application Ser. No. 63/135,543, filed on Jan. 8, 2021, which is incorporated herein by reference in its entirety.

### TECHNICAL FIELD

This document generally describes technology for facilitating the analysis of medical survey data and connecting medical practitioners with patients.

### BACKGROUND

Telemedicine techniques may be used by healthcare workers to evaluate, diagnose, and treat patients at a distance using telecommunications technology. In general, telemedicine may serve as an alternative to in-person patient visits. For example, telemedicine techniques can include robotic surgeries that are facilitated by remote access devices, physical therapy performed using digital monitoring instruments, medical tests being forwarded for interpretation by medical specialists, patient monitoring through the transfer of remote data, online conferences between healthcare workers and patients, and so forth.

### SUMMARY

This document generally describes computer-based technology for facilitating the analysis of medical survey data and connecting medical practitioners with patients. In general, the analysis provided by the technology may operate as a guide for medical practitioners who specialize in diagnosing a patient's medical symptoms and recommending treatment for the patient. One of the issues encountered in the medical field is that the availability of such specialists is limited. Several days, weeks, or even months may pass between a time at which a patient is referred to a specialist, and a time at which the specialist is available to communicate with the patient. Further, an amount of medical data that pertains to the patient is often extensive, and it is often difficult to discern the most relevant details for diagnosing the patient's condition. The technology described in this document includes computerized techniques for efficiently providing an electronic medical survey for completion by a patient, and analyzing and annotating the patient's survey responses such that potentially relevant details are brought to the attention of a specialist, thus saving time and improving results.

The technology described in this document also includes computerized techniques for matching a suitable medical practitioner with a patient, based on the patient's responses to the electronic medical survey. For example, data that represents a pool of medical practitioners can be maintained, and when new medical survey results are received from the patient, medical practitioners that are suitably qualified to diagnose and/or recommend treatment for the patient are selected and notified. A first medical practitioner that responds to the notification, for example, can be assigned to the patient and can receive the patient's responses to the medical survey, thus reducing an amount of time for a patient to be diagnosed and receive a treatment recommendation. After receiving the treatment recommendation, for example, a medical coordination specialist (e.g., a nurse) can be selected to perform various follow-up tasks to assist the patient with receiving treatment, such as ensuring that the patient receives medication, therapy, surgery, a further diagnostic evaluation, or another recommended treatment, to be performed by a medical practitioner other than the medical practitioner that performed the diagnosis and/or provided the recommendation. By separating a medical practitioner that provides diagnoses/recommendations from a medial practitioner that provides treatment, for example, a possible inclination for medical practitioners to overprescribe treatments may be reduced, while focusing on treatments that deliver optimal results for the patient.

In some implementations, a system includes a medical condition data model, the medical condition data model providing associations between medical assessment data and areas of concern; a medical practitioner data repository, the medical practitioner data repository providing a mapping between medical symptoms and medical practitioners; and one or more computing servers configured to perform operations comprising: providing medical survey data to a patient computing device, the medical survey data including (i) a symptom question, and (ii) a plurality of medical assessment questions related to a patient response to the symptom question; receiving, from the patient computing device, the patient response to the symptom question and patient responses to the medical assessment questions; using the medical condition data model to (i) analyze the received patient responses to the medical assessment questions, and (ii) annotate the medical survey data to indicate one or more medical assessment questions having responses that are areas of concern; accessing the medical practitioner data repository to select one or more medical practitioners that specialize in recommending treatment for a symptom that corresponds to the patient response to the symptom question; providing annotated medical survey data to a medical practitioner computing device of at least one of the selected medical practitioners, the annotated medical survey data including the patient responses to the medical assessment questions related to the symptom question, and indications of one or more medical assessment questions having responses that are areas of concern; and receiving, from the medical practitioner computing device, a recommendation for treatment of a patient associated with the annotated medical survey data.

Other implementations of this aspect include corresponding computer-implemented methods, and corresponding apparatuses and computer programs recorded on one or more computer storage devices, each configured to perform the actions of the computer-implemented methods. A system of one or more computers can be configured to perform particular operations or actions by virtue of having software, firmware, hardware, or a combination of them installed on the computers of the system that in operation causes or cause the system to perform the actions. One or more computer programs can be configured to perform particular operations or actions by virtue of including instructions that, when executed by data processing apparatuses, cause the apparatuses to perform the actions.

These and other implementations can include any, all, or none of the following features. The plurality of medical assessment questions related to the patient response to the symptom question can be presented by the patient computing device after input indicating the patient response to the symptom question is received at the patient computing device. The patient response to the symptom question can include an indication of one or more symptoms, and the plurality of medical assessment questions can include a different set of questions related to each indicated symptom. The symptom question can be presented by the patient computing device as a collection of selectable icons, each selectable icon including a different image that depicts a location and an intensity of a different symptom, the patient response to the symptom question being a selection of one or more of the selectable icons. The operations can include providing, to at least two medical practitioner computing devices of at least two of the selected medical practitioners that specialize in recommending treatment for the symptom, a notification that new annotated medical survey data is available for review; and in response to receiving, from a first one of the at least two medical practitioner computing devices, a confirmation to proceed with review of the new annotated medical survey data: providing the new annotated medical survey data for presentation by the first one of the at least two medical practitioner computing devices; and not providing the new annotated medical survey data for presentation by other medical practitioner computing devices. The operations can include, in response to receiving the recommendation for treatment of the patient: accessing the medical practitioner data repository to select one or more medical practitioners that specialize in coordinating treatment for the symptom that corresponds to the patient response to the symptom question; and providing at least some of the medical survey data to a computing device of at least one of the selected medical practitioners that specialize in coordinating treatment of the symptom. The operations can include using the medical condition data model to generate an initial recommendation for treatment of the patient. Providing annotated medical survey data to the medical practitioner computing device can include providing the initial recommendation. The operations can include receiving treatment results data that represent a result of applying the recommendation for treatment to the patient. The operations can include using the treatment results data to update the medical condition data model. The operations can include using the treatment results data to update the medical practitioner data repository. Accessing the medical practitioner data repository to select one or more medical practitioners can include initially selecting medical practitioners that are associated with past treatment results that are positive overall, and not initially selecting medical practitioners that are associated with past treatment results that are not positive overall.

The systems, devices, program products, and processes described throughout this document can, in some instances, provide one or more of the following advantages. By limiting medical assessment questions that are presented to a patient to questions that are related to the patient's symptoms, time can be saved, and an amount of medical survey data that is transmitted, processed, and stored can be reduced. By using illustrative icons with short text descriptions to collect responses to medical assessment questions, the quality of such responses can be improved, and the data collection process can be facilitated. By staging the selection of medical practitioners over time and performing the selection based at least in part of quality of past results, the quality of future results can be improved while providing results in a timely manner.

The details of one or more embodiments are set forth in the accompanying drawings and the description below. Other features and advantages will be apparent from the description and drawings, and from the claims.

### BRIEF DESCRIPTION OF DRAWINGS

FIG. 1 is a conceptual diagram of an example system for facilitating the analysis of medical survey data and connecting medical practitioners with patients.

FIG. 2 shows an example process for facilitating the analysis of medical survey data and connecting medical practitioners with patients.

FIG. 3A-B are conceptual diagrams that show determining selection pools of medical practitioners.

FIGS. 4A-Q show example user interfaces that facilitate patient completion of a medical survey.

FIGS. 5A-H show example user interfaces that facilitate review of a medical survey by a medical practitioner that specializes in recommending patient treatment.

FIGS. 6A-F show example user interfaces that facilitate review of patient information by a medical practitioner that specializes in coordinating patient treatment.

FIG. 7 shows example user interfaces that facilitate review of completed medical surveys by a patient.

FIG. 8 shows an example user interface that facilitates workflow of a medical practitioner.

FIG. 9 is a block diagram of example computing devices that may be used to implement the systems and methods described in this document.

Like reference symbols in the various drawings indicate like elements.

### DETAILED DESCRIPTION

This document generally describes systems, devices, and techniques for facilitating the analysis of medical survey data and connecting medical practitioners with patients. For example, a patient can complete an electronic medical survey which is analyzed and annotated to highlight survey responses that indicate possible areas of concern. Based on the survey responses, for example, the results of the electronic medical survey can be routed to a suitable medical practitioner that specializes in diagnosing and/or recommending treatment for the patient's symptoms. The medical practitioner can easily review the results of the survey, for example, by focusing on medical details indicated by the highlighted survey responses. After contacting the patient, for example, the medical practitioner can provide a diagnosis and/or recommendation for treatment, through a practitioner interface. A suitable coordination specialist (e.g., a nurse) can be assigned to the patient after the treatment recommendation is received, and the coordination specialist can follow up with the patient to ensure that the prescribed treatment is carried out. Optionally, treatment results can be received and can be used to improve processes for annotating surveys and/or selecting suitable medical practitioners.

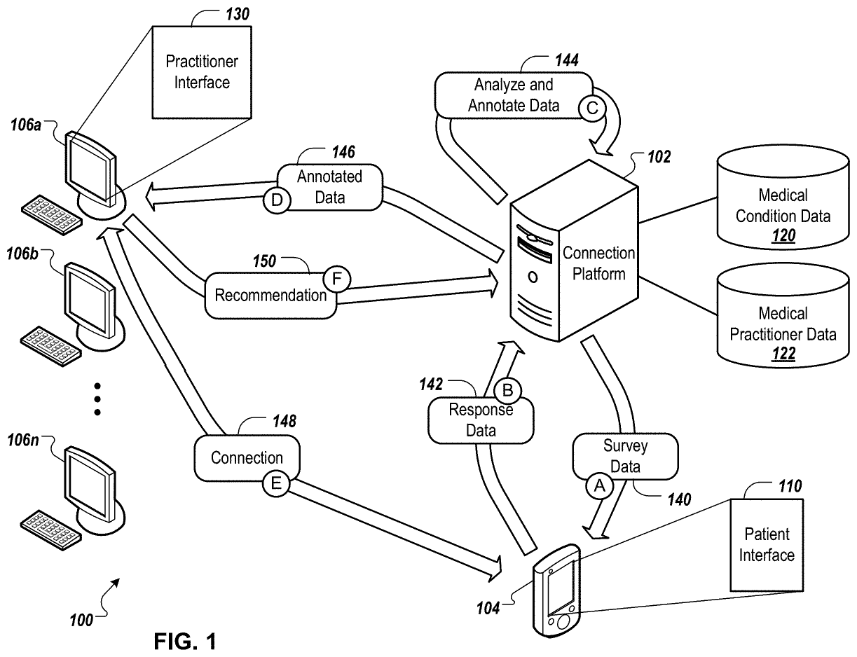

FIG. 1 is a conceptual diagram of an example system 100 for facilitating the analysis of medical survey data and connecting medical practitioners with patients. In the depicted example, the system 100 includes a connection platform 102, one or more patient computing devices 104, and one or more medical practitioner computing devices 106. The connection platform 102, for example, can include various forms of computing servers including, but not limited to network servers, web servers, application servers, or other suitable computing servers. The connection platform 102 can access and maintain data from various data sources, including a medical condition data source 120 and a medical practitioner data source 122, for example. The data sources 120, 122, for example, can include databases, file systems, computer memory, and/or other suitable data sources. Each of the patient computing device(s) 104 and the medical practitioner computing device(s) 106, for example, can be a mobile computing device (e.g., smartphone, tablet, laptop, personal digital assistant, etc.), a stationary computing device (e.g., personal computer, kiosk, etc.), or another suitable computing device. The patient computing device(s) 104, the medical practitioner computing device(s) 106, and the connection platform 102, for example, can communicate with each other over one or more communication networks (not shown), including a LAN (local area network), a WAN (wide area network), and/or the internet.

FIG. 1 also illustrates an example process flow for facilitating the analysis of medical survey data and connecting medical practitioners with patients, shown in stages (A) to (F). Stages (A) to (F) may occur in the illustrated sequence, or they may occur in a sequence that is different than in the illustrated sequence, and/or two or more stages (A) to (F) may be concurrent.

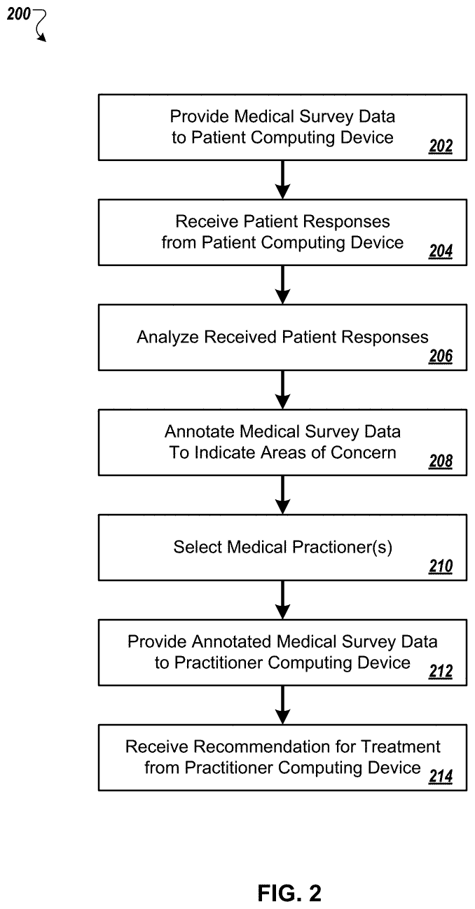

Referring now to FIG. 2, an example process 200 is shown for facilitating the analysis of medical survey data and connecting medical practitioners with patients. The process 200 can be performed by components of the system 100, for example, and will be described with reference to FIG. 1. However, other systems may be used to perform the same or similar process.

Medical survey data can be provided to a patient computing device (202). Referring again to FIG. 1, for example, during stage (A), the connection platform 102 can provide medical survey data 140 to the patient device 104. For example, the patient computing device 104 can execute an application (e.g., a proprietary application, a web browser, etc.) that renders and displays a patient interface 110, through which a patient can initiate and complete one or more medical surveys provided by the connection platform 102. The medical survey data 140, for example, can be used by the patient interface 110 to present an electronic medical survey that includes one or more symptom questions, and a plurality of medical assessment questions related to a patient response to each symptom question (e.g., through visual and/or audible prompts). The patient interface 110 can be used to facilitate the completion of the electronic medical survey(s) represented by the medical survey data 140, for example, by collecting responses to the symptom question(s) and the related medical assessment questions provided by the patient.

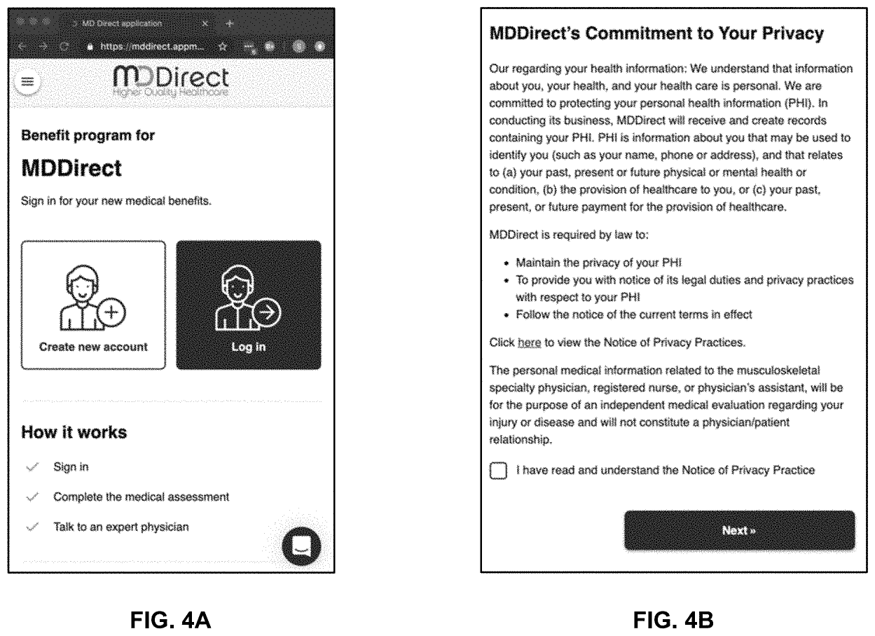

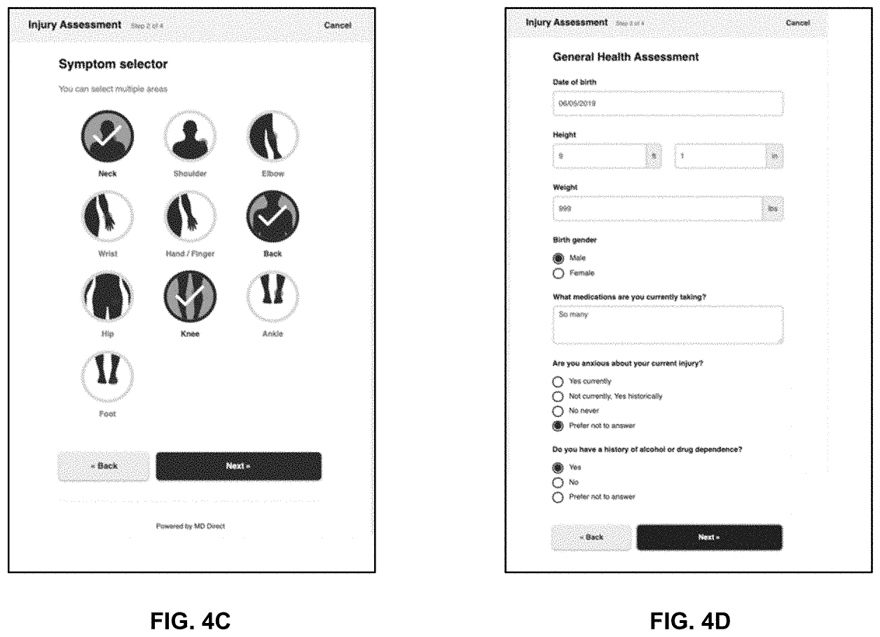

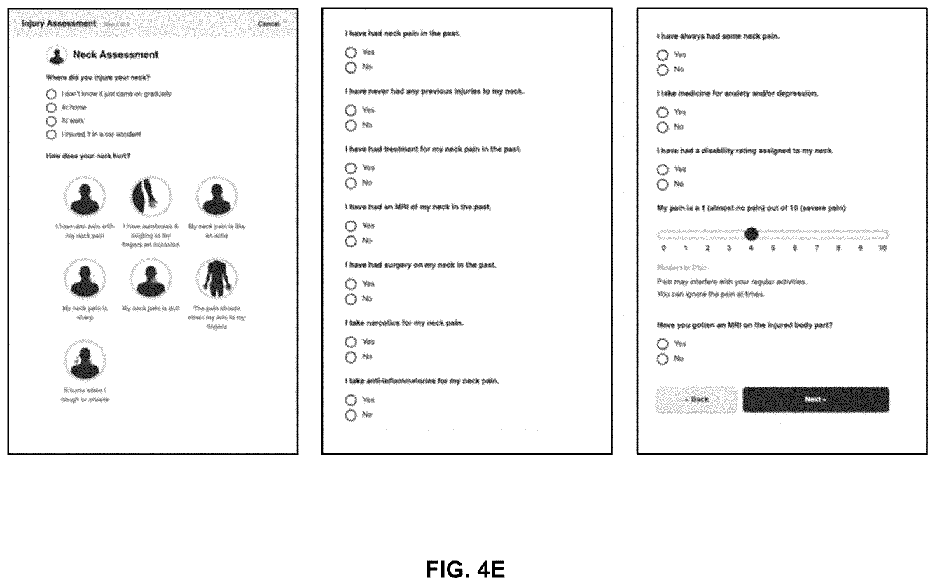

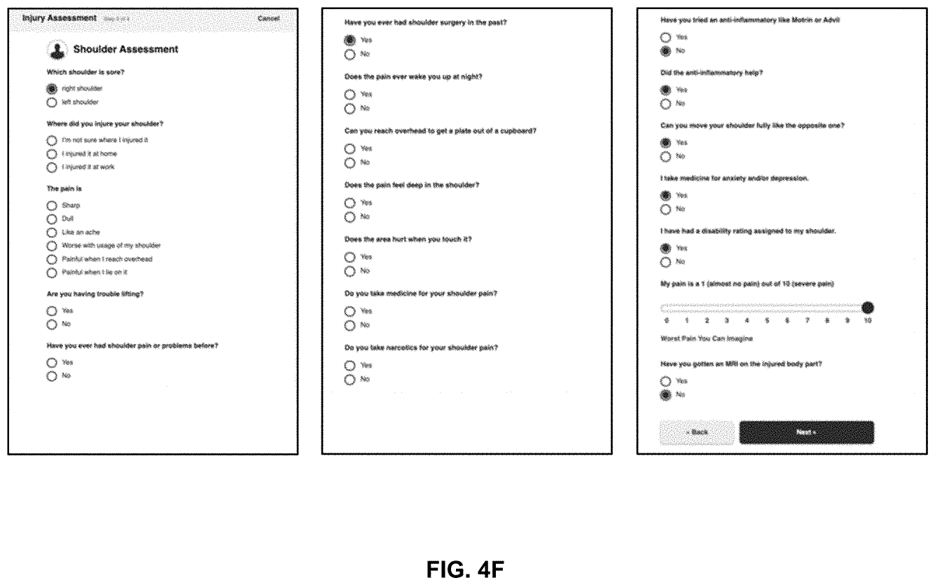

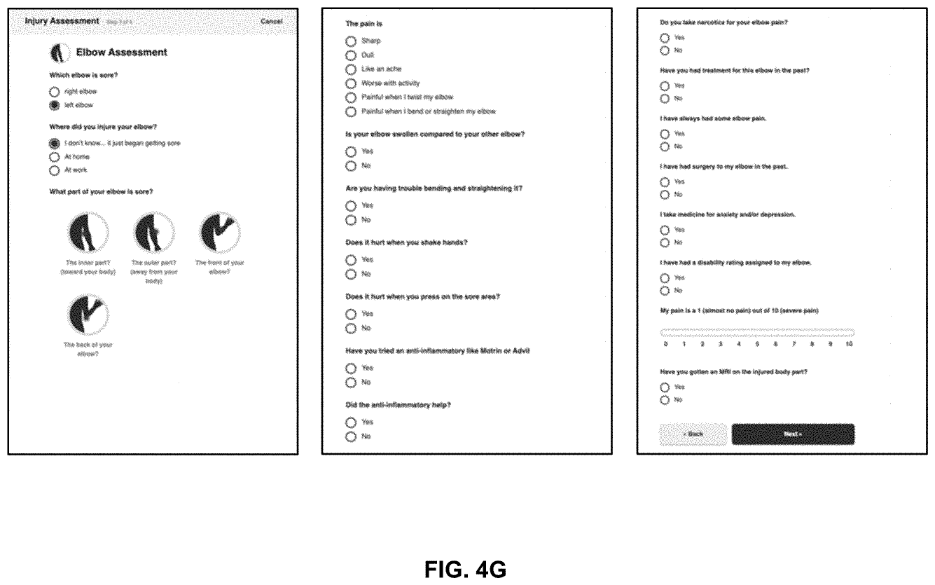

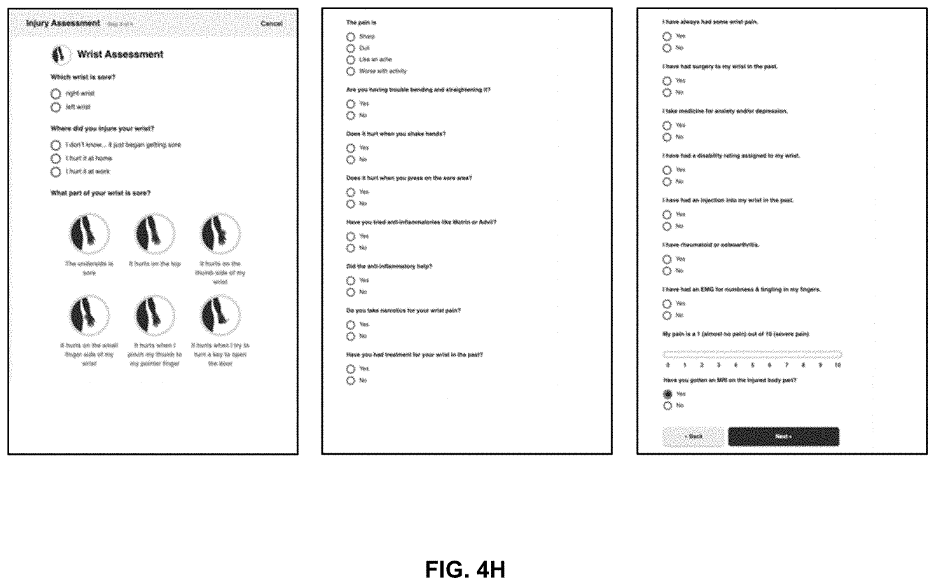

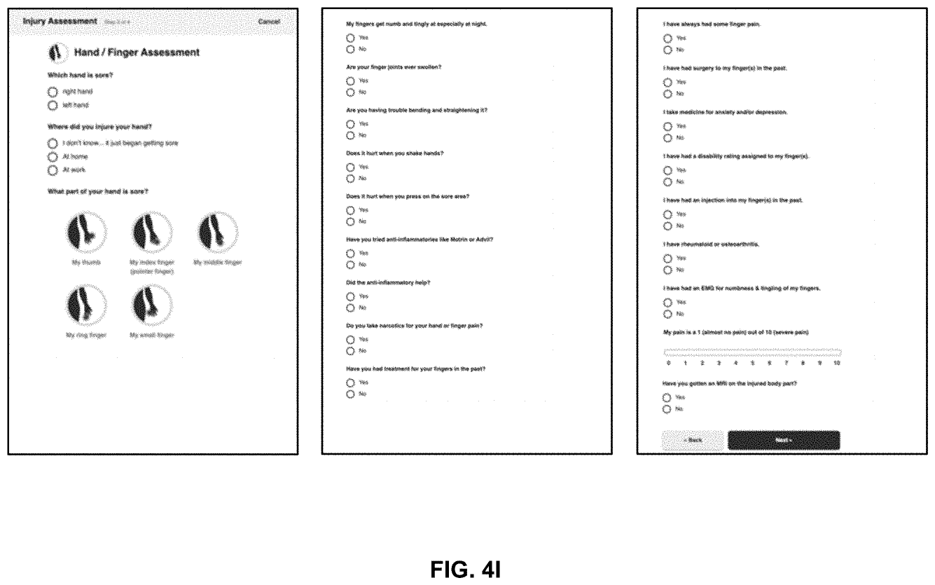

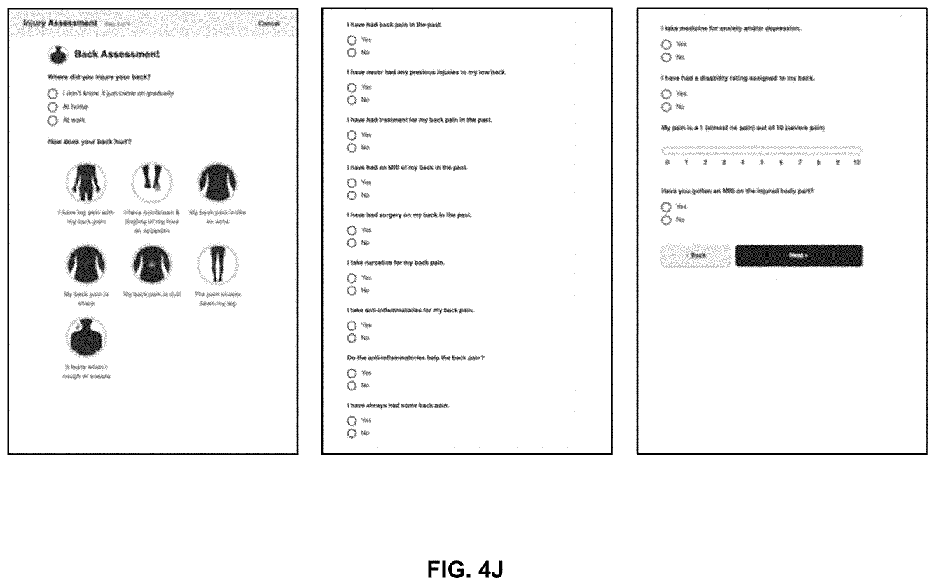

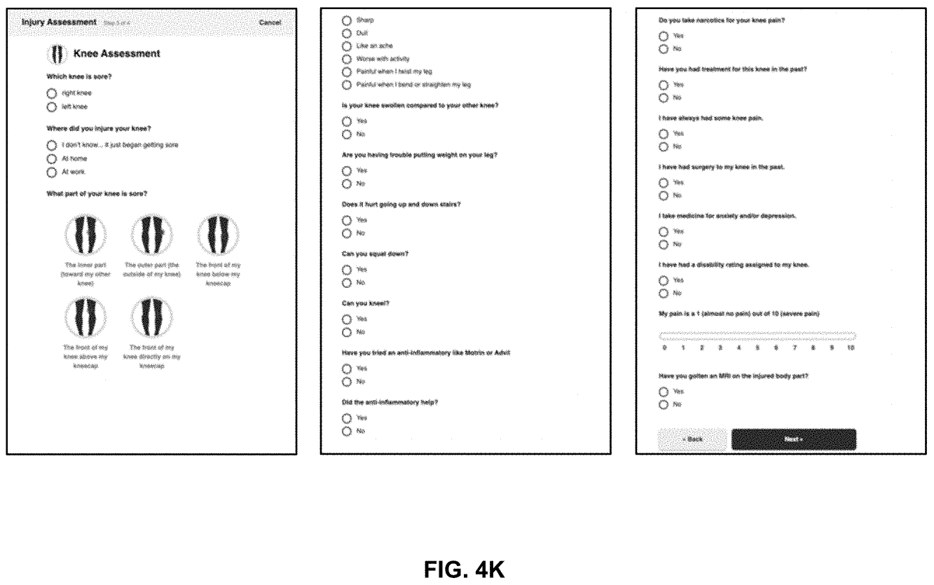

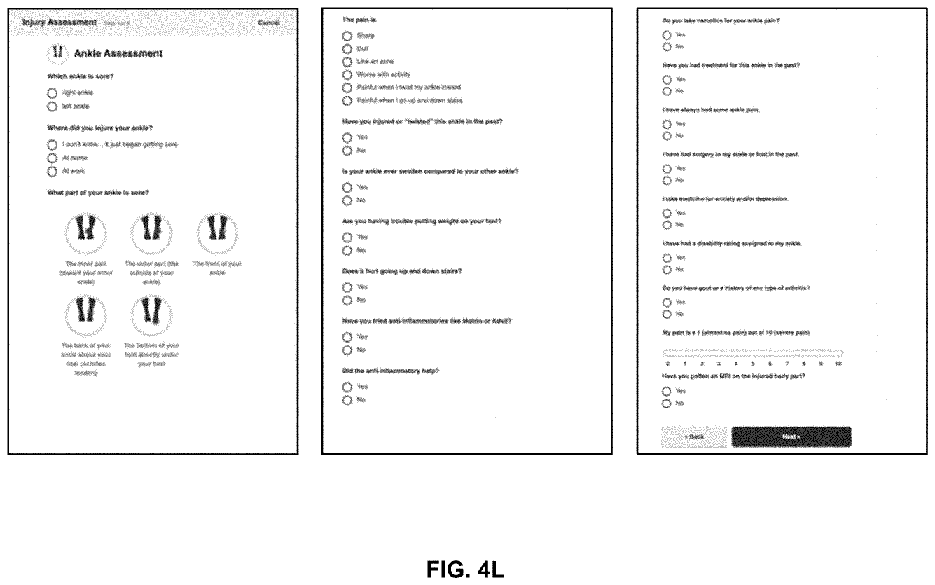

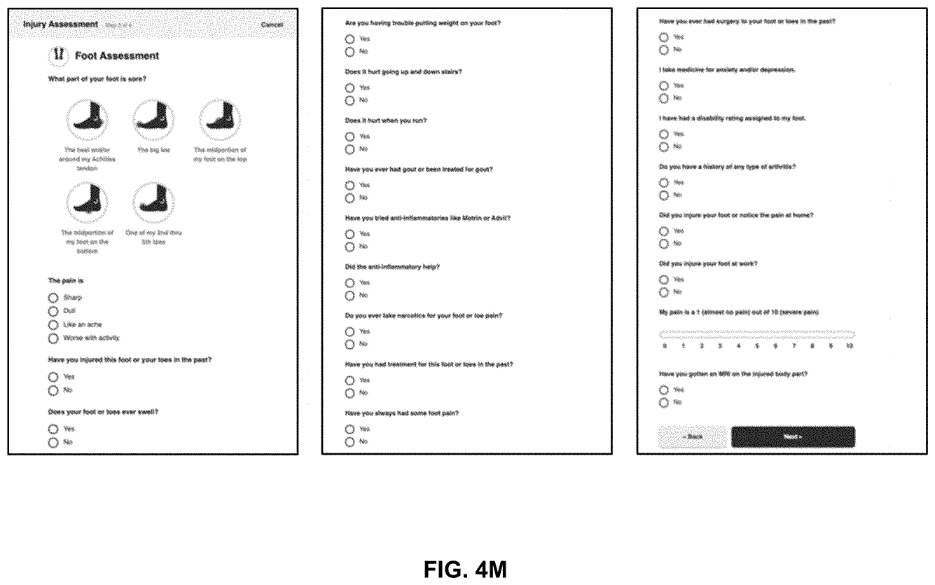

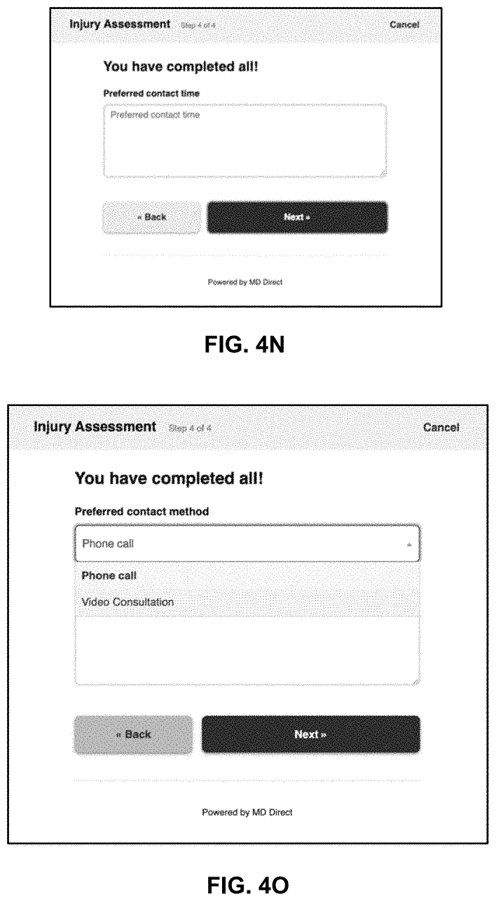

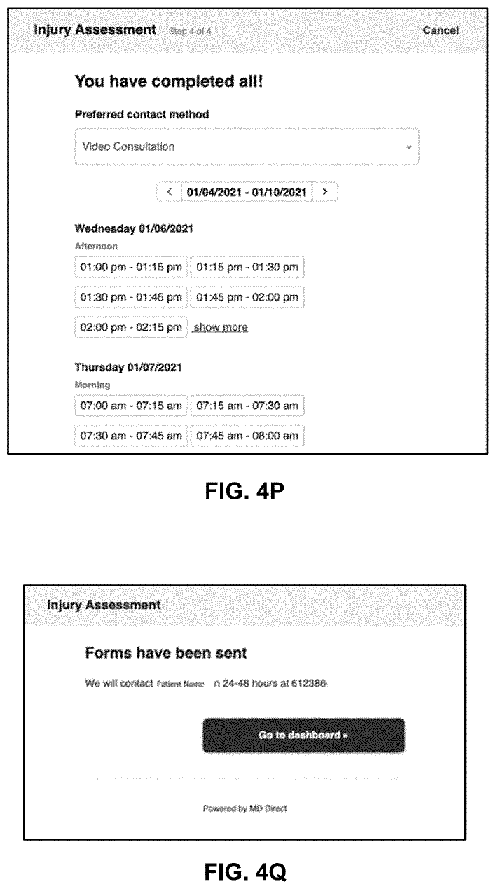

Referring now to FIGS. 4A-Q, example user interfaces that facilitate patient completion of a medical survey (e.g., display screens of the patient interface 110 presented by the patient computing device 104, shown in FIG. 1) are shown. FIG. 4A, for example, shows a starting interface that facilitates the creation of a new account, or logging into an existing account by a patient. The starting interface may also include a high-level overview of features provided by a medical survey application/connection platform. FIG. 4B, for example, shows a privacy information interface. The privacy information interface may include a description of policies to be followed with respect to the collection and use of medical data provided by the patient.

Referring now to FIG. 4C, for example, an assessment interface is shown for presenting a symptom question and for receiving a patient response to the symptom question. In some implementations, a symptom question may be presented by a computing device as a collection of selectable icons, each selectable icon including a different image that depicts a location and an intensity of a different symptom. For example, the assessment interface includes a first selectable icon that depicts a first symptom (e.g., a red dot representing pain is placed on an image of a patient's neck) along with a short text description of the first symptom (e.g., “Neck”), a second selectable icon that depicts a second symptom (e.g. a red dot representing pain is placed on an image of a patient's shoulder) along with a short text description of the first symptom (e.g., “Shoulder”), a third selectable icon that depicts a third symptom (e.g., a red dot representing pain is placed on an image of a patient's elbow) along with a short text description of the third symptom (e.g., “Elbow”), and so forth. A patient's response to the symptom question may be expressed as a selection of one or more of the selectable icons. In the present example, the “Neck,” “Back,” and “Knee” icons have been selected through the assessment interface to indicate which symptoms are currently being experienced by the patient. After providing the patient response to the symptom question, for example, a “Next” control can be selected to navigate to a general health assessment interface.

Referring to FIG. 4D, for example, a general health assessment interface is shown for facilitating the collection of general health information about a patient. For example, the general health assessment interface can facilitate the collection of general health information such as age, height, weight, gender, current medications, and so forth. After providing the response to the symptom question (e.g., selecting one or more icons that depict various symptoms), and after providing the general health assessment information, for example, a “Next” control can be selected to navigate to various interfaces for collecting responses related to the patient response(s) to the symptom question. Each of the interfaces shown in FIGS. 4E-M, for example, can present different sets of medical assessment questions related to different symptoms, and collect patient responses to the medical assessment questions. FIG. 4E, for example, shows interfaces used for assessing neck pain. FIG. 4F, for example, shows interfaces used for assessing shoulder pain. FIG. 4G, for example, shows interfaces used for assessing elbow pain. FIG. 4H, for example, shows interfaces used for assessing wrist pain. FIG. 4I, for example, shows interfaces used for assessing hand/finger pain. FIG. 4J, for example, shows interfaces used for assessing back pain. FIG. 4K, for example, shows interfaces used for assessing knee pain. FIG. 4L, for example, shows interfaces used for assessing ankle pain. FIG. 4M, for example, shows interfaces used for assessing foot pain. Interfaces for presenting and collecting responses to other sets of medical assessment questions related to other symptoms are possible.

In general, a first set of medical assessment questions can be provided through an interface after receiving input indicating selection of a first selectable icon that depicts a first symptom (e.g., neck pain), a second set of medical assessment questions can be provided through an interface after receiving input indicating selection of a second selectable icon that depicts a second symptom (e.g., shoulder pain), a third set of medical assessment questions can be provided through an interface after receiving input indicating selection of a third selectable icon that depicts a third symptom (e.g., elbow pain), and so forth. In some implementations, only the sets of medical assessment questions that are related to responses to the symptom question are provided through interfaces. For example, after receiving input indicating selection of only the first symptom (e.g., neck pain), only the interfaces used for assessing the first symptom (e.g., shown in FIG. 4E) may be provided, and the other assessment interfaces (e.g., shown in FIGS. 4F-M) may not be provided. As another example, after receiving input indicating selection of only the first and second symptoms (e.g., neck pain and shoulder pain), only the interfaces used for assessing the first and second symptoms (e.g., shown in FIGS. 4E and 4F) may be provided, and the other assessment interfaces (e.g., shown in FIGS. 4G-M) may not be provided. By limiting the sets of medical assessment questions that are presented to a patient to questions that are related to a symptom response, for example, time can be saved, and an amount of medical survey data that is transmitted and stored can be reduced.

In some implementations, an interface for presenting a set of medical assessment questions related to a symptom and collecting responses to the questions may include a collection of selectable icons, each selectable icon including a different image that depicts a location and an intensity of a different specific description of the symptom. Referring again to FIG. 4E, for example, the interfaces used for assessing neck pain include a first selectable icon that depicts a first specific description (e.g., a red dot representing pain is placed on an image of a patient's neck and another red dot representing pain is placed on an image of the patient's arm) along with a short text description (e.g., “I have arm pain with my neck pain”), a second selectable icon that depicts a second specific description (e.g., a blue dot representing numbness is placed on an image of a patient's hand) along with a short text description (e.g., “I have numbness & tingling in my fingers on occasion”), a third selectable icon that depicts a third specific description (e.g., a red dot representing pain is placed on an image of a patient's neck) along with a short text description (e.g., “My neck pain is like an ache”), and so forth. A patient's response to the medical assessment question may be indicated through a selection of one or more of the selectable icons, for example. In the present example, the patient's response to the medical assessment question of “How does your neck hurt?” includes the selection of the “I have arm pain with my neck pain” and the “The pain shoots down to my arm to my fingers” icons. Using illustrative icons with short text descriptions to collect responses to medical assessment questions, for example, can improve the quality of such responses and can facilitate the data collection process, as patients can quickly and easily identify appropriate responses to the questions.

After responses to the medical assessment questions have been received (e.g., through one or more of the interfaces shown in FIGS. 4E-M), the computing device 104 (shown in FIG. 1) can notify a patient that a medical survey is complete through the patient interface 110 (also shown in FIG. 1). Referring to FIG. 4N, for example, a completion interface is shown for presenting a message that the medical survey has been completed, and for presenting a control through which a patient may provide a preferred contact time at which the patient is available for contact by a medical practitioner.

In some implementations, a completion interface may include one or more controls for facilitating selection of a contact method by a patient and/or scheduling of consultation session. Referring to FIG. 4O, for example, a completion interface is shown for presenting a message that the medical survey has been completed, and for presenting a control through which the patient can select a preferred contact method for contact by a medical practitioner. Selectable options for the preferred contact method, for example, can include a phone call, a video consultation, and other suitable contact methods. In the present example, the patient has selected a video consultation as the preferred contact method. After the preferred contact method has been selected by the patient, for example, the patient interface 100 (shown in FIG. 1) can provide a scheduling interface that includes one or more scheduling controls for selecting a time at which the medical practitioner is to contact the patient. Referring to FIG. 4P, for example, a scheduling interface is shown for presenting a calendar control and multiple different selectable time block controls, each time block control corresponding to a block of time at which the medical practitioner is available for a consultation (e.g., based on the medical practitioner's schedule). The patient can select a preferred block of time using the scheduling interface, for example.

Referring again to FIG. 2, patient responses can be received from the patient computing device (204). After the preferred contact time has been provided and the “Next” control has been selected (shown in FIG. 4N), or after the preferred contact method and preferred block of time has been selected (shown in FIGS. 4O-P) for example, a notification interface can be presented (shown in FIG. 4Q), which notifies a patient that the provided responses to the symptom question and the medical assessment questions have been sent to the connection platform 102 (shown in FIG. 1). The notification interface can also include a time window (e.g., 24-48 hours, or another time window) in which the patient may expect contact from a suitable medical practitioner who specializes in diagnosing the patient's symptoms, for example. As another example, the notification interface can include a confirmation of the preferred time block selected by the patient.

Referring again to FIG. 1, for example, during stage (B), the connection platform 102 can receive response data 142 provided by the patient computing device 104. The response data 142, for example, can include the patient response to the symptom question, and patient responses to the medical assessment questions related to the patient's response to the symptom question. For example, if the patient had selected the icon that depicts the “Neck Pain” symptom (e.g., shown in FIG. 4C), the response data 142 can include data that indicates the patient selection of the icon, and the patient responses to the related medical assessment questions (e.g., responses to the questions shown in FIG. 4E). The response data 142, for example, can also include data that indicates the patient's preferred contact time (e.g., the time provided using the interface shown in FIG. 4N).

Referring again to FIG. 2, the received patient responses can be analyzed (206) and annotated (208) to indicate one or more areas of concern. As shown in FIG. 1, for example, during stage (C), the connection platform 102 can analyze and annotate data 144. Analyzing and annotating data, for example, can generally include analyzing the received response data 142 in the context of medical condition data maintained by the medical condition data source 120, and annotating the data 142 such that possible areas of concern are emphasized, thus improving the usefulness of such data by medical practitioners that specialize in recommending treatment for symptoms. For example, the connection platform 102 can access the medical condition data source 120 and reference a medical condition data model that provides associations between medical assessment data and areas of concern. In general, areas of concern may include medical assessment questions and responses (and/or combinations of questions/responses) that tend to correspond to particular diagnoses and/or recommended treatment from medical practitioners when the questions/responses occur in received medical surveys. For example, the medical condition data source 120 can include an aggregation of previously received response data 142 from various different patients (e.g., each patient using a different patient computing device 104), and the connection platform 102 can use machine learning techniques to associate particular responses (and/or response combinations) with particular diagnoses made by medical practitioners when analyzing the response data. As another example, the medical condition data source 120 can include expert associations between particular responses (and/or response combinations) and particular diagnoses. In the present example, the medical condition data model provided by the medical condition data source 120 can be used by the connection platform 102 to analyze the patient responses to the medical assessment questions in the response data 142 received from the patient computing device 104, and to annotate the response data 142 to indicate one or more medical assessment questions having responses that are areas of concern.

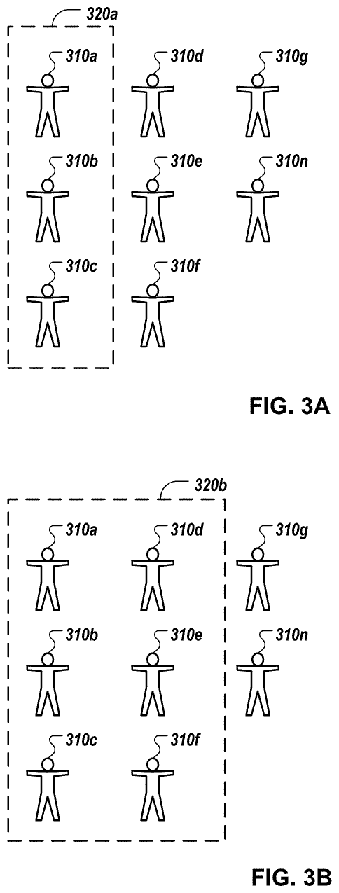

Referring again to FIG. 2, one or more medical practitioners can be selected (210). As shown in FIG. 1, for example, the connection platform 102 can access the medical practitioner data source 122 and reference data that provides a mapping between various medical symptoms and various medical practitioners. Referring now to FIGS. 3A-B, for example, conceptual diagrams are shown for determining selection pools of medical practitioners. In general, determining a selection pool of medical practitioners may include identifying patient response(s) to a symptom question in received survey data, and identifying one or more medical practitioners that specialize in diagnosing the symptom(s) and/or recommending treatment for the symptom(s) indicated in the patient response(s). For example, if the patient had selected the icon that depicts the “Neck Pain” symptom (e.g., shown in FIG. 4C), the connection platform 102 can identify one or more medical practitioners represented in the medical practitioner data 122 that specialize in diagnosing and/or recommending treatment for neck pain. In the present example, a group of medical practitioners 320a (e.g., including medical practitioners 310a-c, shown in FIG. 3A) can be selected for diagnosing and/or recommending treatment for the patient's symptom indicated in the received response data 142 (shown in FIG. 1), based at least in part on mapped specialties of the medical practitioners.

In some implementations, accessing a medical practitioner data repository to select one or more medical practitioners may include initially selecting medical practitioners that are associated with past treatment results that are generally more positive, and not initially selecting medical practitioners that are associated with past treatment results that are generally less positive. For example, the group of medical practitioners 320a (e.g., including medical practitioners 310a-c, shown in FIG. 3A) may each be associated with generally positive results for diagnosing a particular symptom and/or recommending treatment for the symptom. Medical practitioners 310d-f, however, may each be associated with relatively less positive results, and medical practitioners 310g-n may not be qualified to diagnose and/or recommend treatment for the symptom (e.g., based on a mapping of medical specialties and/or poor past results). In the present example, the group of medical practitioners 320a can be initially selected for diagnosing and/or recommending treatment for a patient's “Neck Pain” symptom, based on generally positive past results. After a threshold period of time has transpired (e.g., six hours, twelve hours, a day, or another suitable threshold period of time), for example, if none of the medical practitioners in the group of medical practitioners 320a have indicated a diagnosis and/or recommended treatment for the patient (e.g., through use of practitioner interface 130, shown in FIG. 1), the connection platform 102 can subsequently select an expanded group of medical practitioners 320b (e.g., including medical practitioners 310a-f, shown in FIG. 3B) for diagnosing and/or recommending treatment for the patient's symptom indicated in the received response data 142 (shown in FIG. 1). In the present example, medical practitioners 310g, 310n, can be excluded from the selection of medical practitioners. By staging the selection of medical practitioners over time and performing the selection based at least in part of quality of past results, for example, the quality of future results can be improved while providing results in a timely manner.

In some implementations, a notification that new annotated medical survey data is available for review may be provided to a medical practitioner computing device of a selected medical practitioner that specializes in diagnosing and/or recommending treatment for a symptom. Referring again to FIG. 1, for example, such a notification can be provided to one or more medical practitioner computing devices 106a-n (e.g., corresponding to medical practitioners 310a-n, shown in FIGS. 3A and 3B). The notifications, for example, can be presented by the respective medical practitioner computing devices 106a-n through a user interface (e.g., practitioner interface 130, shown in FIG. 1). For example, each of the medical practitioner computing devices 106a-n can execute applications (e.g., proprietary applications, web browsers, etc.) that render and display practitioner interface 130, through which a medical practitioner can interact with the connection platform 102.

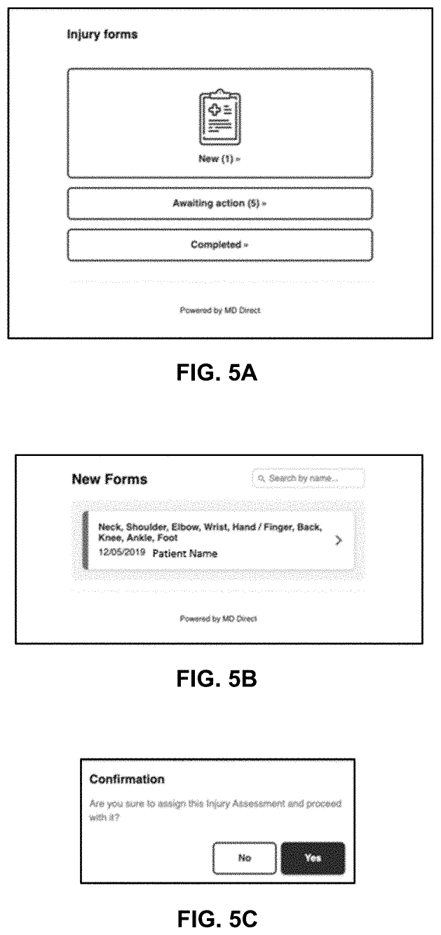

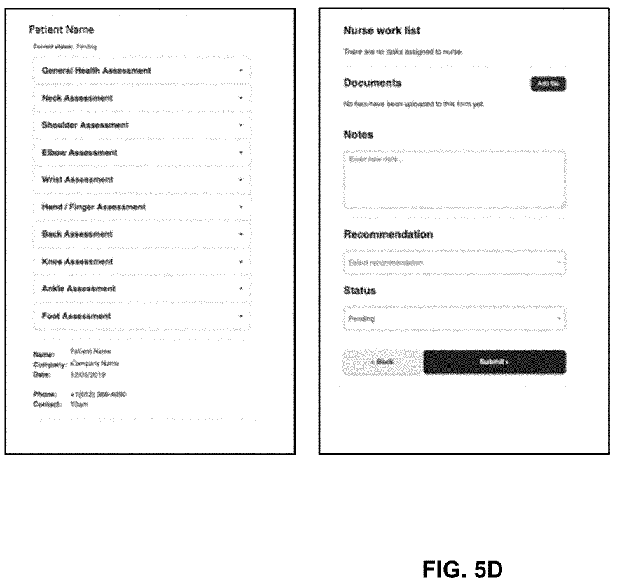

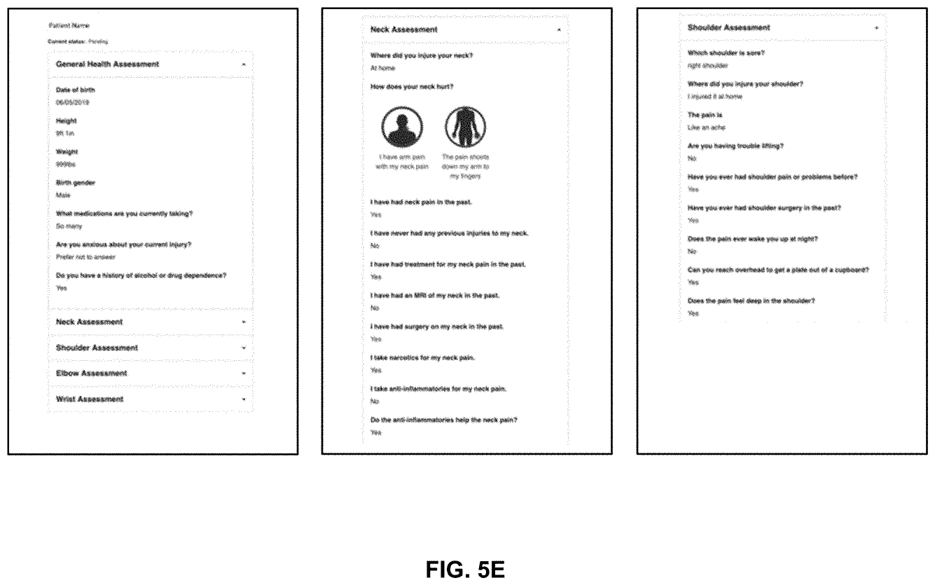

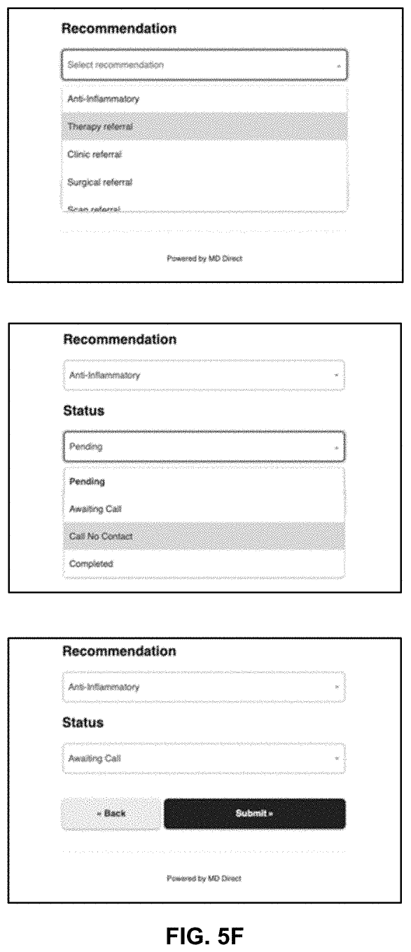

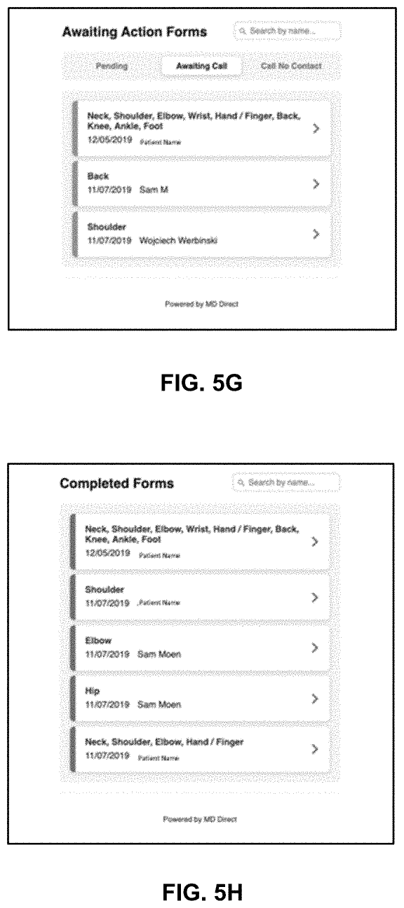

Referring now to FIGS. 5A-H, example user interfaces that facilitate review of a medical survey by a medical practitioner that specializes in diagnosing and/or recommending patient treatment (e.g., display screens of the practitioner interface 130) are shown. FIG. 5A, for example, shows a dashboard interface that presents notifications of new medical surveys that are available for review (e.g., surveys that have been completed by patients but not yet assigned to and/or accepted by a medical practitioner for review), representations of medical surveys that are awaiting action (e.g., surveys that have been completed by patients and have been assigned to and/or accepted by the medical practitioner for review), and representations of medical surveys for which review has been completed by the medical practitioner. In response to selection of the “New Survey” control, for example, a new medical survey interface (shown in FIG. 5B) can be presented, in which an overview of a completed medical survey is presented, including patient symptoms addressed in the survey, a survey completion date, a patient name, and other relevant data. If the medical practitioner chooses to review a completed medical survey, for example, the medical practitioner can select a control that represents the completed survey. In response to selection of the control that represents the completed medical survey, for example, a confirmation interface (shown in FIG. 5C) can be presented, through which the medical practitioner can confirm acceptance of the medical survey for review.

In some implementations, a notification that new annotated medical survey data is available for review may be provided to at least two medical practitioner computing devices of at least two of the selected medical practitioners that specialize in diagnosing and/or recommending treatment for a symptom. Referring again to FIG. 1, for example, if such a notification is provided to medical practitioner computing devices 106a, 106b (e.g., corresponding to medical practitioners 310a, 310b, shown in FIGS. 3A and 3B), the notifications can be presented by the respective medical practitioner computing devices 106a, 106b through respective practitioner interfaces 130 (shown in FIG. 1). In response to receiving a confirmation to proceed with review of new annotated medical survey data from one of the computing devices 106a, 106b, for example, the survey data can be provided for presentation by the computing device from which the confirmation was received, but not the other computing device. For example, a medical practitioner that first responds to the notification provided by the connection platform 102 (e.g., medical practitioner 310a using computing device 106a to interact with the confirmation interface shown in FIG. 5C) can be assigned by the connection platform 102 to a patient associated with new medical survey data, and the new annotated medical survey data can be provided to the medical practitioner's computing device. As another example, the connection platform 102 can assign a single medical practitioner to a patient associated with new medical survey data (e.g., only medical practitioner 310a using computing device 106a), and can provide the new annotated medical survey data to a computing device of the assigned medical practitioner.

Referring again to FIG. 2, annotated medical survey data can be provided to a medical practitioner computing device of at least one of the selected medical practitioners (212). As shown in FIG. 1, for example, during stage (D), the connection platform 102 can provide annotated medical data 146 to the medical practitioner computing device 106a. The annotated medical data 146, for example, can include patient responses to the medical assessment questions in the medical survey (e.g., the response data 142), and indications of one or more medical questions having responses that are areas of concern (e.g., as determined by the connection platform 102).

Referring now to FIG. 5D, for example, an overview interface is shown for providing general information associated with a patient, links to portions of the patient's completed medical survey, and other related information. In the present example, the overview interface shown in FIG. 5D includes a patient's name, contact information (e.g., phone number, e-mail address, etc.), and preferred contact time (e.g., the contact time provided using the interface shown in FIG. 4N). The example overview interface shown in FIG. 5D also includes a link to general health information for the patient (e.g., provided using the general health interface shown in FIG. 4D), and links to responses to sets of medical assessment questions related to various different symptoms (e.g., provided using one or more of the interfaces shown in FIGS. 4E-M). If a medical practitioner that specializes in facilitating/coordinating patient treatment (e.g., a nurse, a case worker, etc.) has been assigned to the patient and/or case, for example, the overview interface can display a work list for the coordinating specialist. The example overview interface shown in FIG. 5D also includes controls for the medical practitioner to associate documents with the patient's completed medical survey, to enter notes, to update a case status, and/or to select a recommendation.

In some implementations, providing annotated medical survey data to a medical practitioner computing device may include providing an initial diagnosis and/or recommendation. Referring again to FIG. 1, for example, when analyzing and annotating data 144, the connection platform 102 can access the medical condition data source 120 and reference a medical condition data model that provides associations between medical assessment data and recommendations that had been provided in other medical cases having successful outcomes. The medical condition data model can be used to generate an initial recommendation for treatment of the patient, which can be provided to a medical practitioner, for example, through the overview interface shown in FIG. 5D. The medical practitioner may consider the generated recommendation, for example, when reviewing the patient's survey response data, and may choose to confirm the recommendation or provide another recommendation.

Referring now to FIG. 5E, for example, a detailed interface is shown for providing annotated medical survey data. For example, if a medical practitioner selects a link to general health information for a patient (e.g., age, height, weight, gender, current medications, and so forth), the corresponding information can be presented to the medical practitioner through the interface shown in FIG. 5E. Similarly, if the medical practitioner selects a link to responses to a set of questions related to a symptom (e.g., a neck pain assessment, a shoulder pain assessment, an elbow pain assessment, a wrist pain assessment, and so forth), the corresponding information can be presented to the medical practitioner through the interfaces shown in FIG. 5E. In the present example, patient responses for a neck pain assessment and a shoulder pain assessment are shown, with visual indications for medical assessment questions having responses that are areas of concern (e.g., indicated by a highlight color, a special font, or another suitable graphical indication), as determined by the connection platform 102.

Referring again to FIG. 1, for example, during stage (E), a connection 148 between a medical practitioner and a patient can be facilitated. For example, the medical practitioner can place a telephone call to the patient using the patient's contact information (e.g., shown in FIG. 5D). As another example, the connection platform 102 can facilitate the connection 148 using medical practitioner/patient contact information stored by the platform. During a communication session between the medical practitioner and the patient (e.g., including voice, video, and/or text communications), the medical practitioner can elicit further details that pertain to the patient's symptoms, based on the patient's responses to the medical survey. The medical practitioner may choose to focus on the medical assessment questions having responses that have been flagged as possible areas of concern, for example, to elicit pertinent details for making a diagnosis and/or recommendation. For some cases, for example, the medical practitioner may choose not to contact the patient before making a diagnosis and/or recommendation (e.g., cases in which a proper diagnosis is clear based on the annotated data 146).

Referring again to FIG. 2, for example, a diagnosis and/or recommendation for treatment of a patient can be received from the medical practitioner computing device (214). As shown in FIG. 1, for example, during stage (F), the connection platform 102 can receive recommendation data 150 from the medical practitioner computing device 106a. After a medical practitioner has reviewed the patient's annotated medical survey data and has optionally contacted the patient, for example, the medical practitioner can use a recommendation interface (e.g., shown in FIG. 5F) to provide a recommendation for patient treatment (e.g., anti-inflammatory treatment, a therapy referral, a clinic referral, a surgical referral, a scan referral, or another suitable treatment). In the present example, the medical practitioner uses the recommendation interface to select an anti-inflammatory as a recommended treatment for the patient. The medical practitioner may also use the recommendation interface shown in FIG. 5F, for example, to update a case status (e.g., pending, awaiting call, call with no contact, completed, or another suitable status). In the present example, the medical practitioner updates the case status to “awaiting call,” to indicate that contact with the patient has yet to occur. The initial recommendation selected by the medical practitioner may change after contact with the patient, for example. FIG. 5G shows an example interface for presenting summary information (e.g., patient name, survey completion date, general symptoms, etc.) about cases that are awaiting action (e.g., cases with status of pending, awaiting call, and call with no contact). To view the information about cases that are awaiting action, for example, the medical practitioner can select the “awaiting action” control from the medical practitioner dashboard interface (shown in FIG. 5A). FIG. 5H shows an example interface for presenting summary information (e.g., patient name, survey completion date, general symptoms, etc.) about completed cases. To view the information about completed cases, for example, the medical practitioner can select the “Completed” control from the medical practitioner dashboard interface (shown in FIG. 5A).

In some implementations, in response to receiving a recommendation for treatment of a patient, one or more medical practitioners that specialize in facilitating and/or coordinating patient treatment may be selected, and at least some of the medical survey data may be provided to a computing device of at least one of the selected medical practitioners. For example, some of the medical practitioner computing devices 106a-n can be operated by medical practitioners that specialize in facilitating and/or coordinating patient treatment (e.g., nurses, case managers, etc.). Such coordination specialists can be selected by the connection platform 102, for example, to follow up with a patient and to ensure that the recommendation represented in the recommendation data 150 is carried out. In general, techniques for selecting such coordination specialists may be similar to the techniques described with respect to the selection of one or more medical practitioners that specialize in recommending treatment for a symptom that corresponds to the patient response to the symptom question (e.g., FIG. 2, box 210).

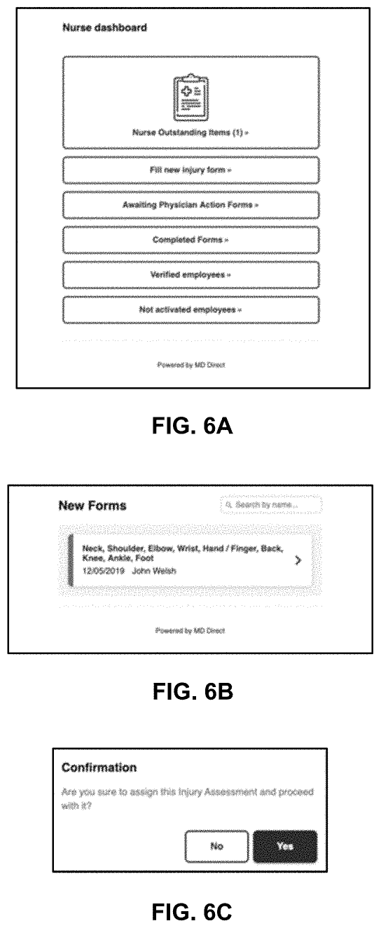

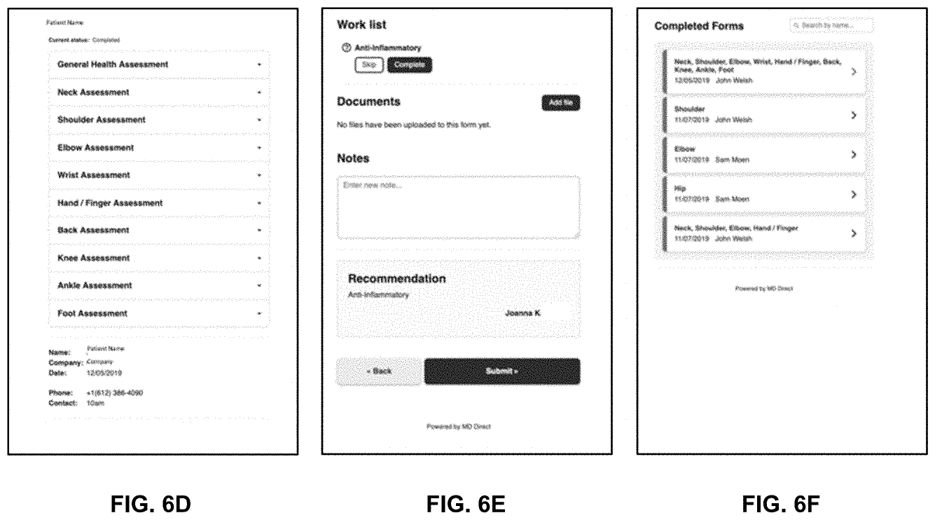

Referring now to FIGS. 6A-F, example user interfaces that facilitate review of patient information by a medical practitioner that specializes in coordinating patient treatment are shown. FIG. 6A, for example, shows a dashboard interface that presents notifications of new medical cases (e.g., surveys that have been completed by patients and for which a medical practitioner has completed a recommendation). In response to selection of the “New Outstanding Item” control, for example, a new case interface (shown in FIG. 6B) can be presented, in which an overview of a new case is presented, included patient symptoms addressed in the corresponding medical survey, a survey completion date, a patient name, and other relevant data. If a coordination specialist selects a new case, for example, a confirmation interface (shown in FIG. 6C) can be presented, through which the coordination specialist can confirm acceptance of the new case. Similar to techniques for providing medical survey data to medical practitioners that specialize in recommending patient treatment, for example, assigning a new case to a coordination specialist can include the connection platform 102 providing notifications of the new case to multiple different coordination specialists that specialize in coordinating treatment for a patient's symptom, and assigning the case to the first coordination specialists that confirms acceptance of the case. As another example, the connection platform 102 can assign a single coordination specialist to a new case.

Referring now to FIG. 6D, for example, an overview interface is shown for providing general information associated with a patient, links to portions of the patient's completed medical survey, and other related information. In the present example, the overview interface shown in FIG. 6D includes a patient's name, contact information (e.g., phone number, e-mail address, etc.), and preferred contact time (e.g., the contact time provided using the interface shown in FIG. 4N), and links to responses to sets of medical assessment questions related to various different symptoms (e.g., provided using one or more of the interfaces shown in FIGS. 4E-M).

Referring now to FIG. 6E, for example, a work list interface is shown for listing one or more work items to be completed by a coordination specialist for an accepted case. In the present example, a work item to be completed is an anti-inflammatory follow-up, which corresponds to an anti-inflammatory treatment recommended by a medical practitioner that specializes in recommending patient treatment. After performing the work item, for example, the coordination specialist can submit the work item. FIG. 6F shows an example interface for presenting summary information (e.g., patient name, survey completion date, general symptoms, etc.) about cases that the coordination specialist has completed (e.g., for which all work items have been completed). To view the information about completed cases, for example, the coordination specialist can select the “Completed” control from the coordination specialist dashboard interface (shown in FIG. 6A).

Referring now to FIG. 7, for example, user interfaces that facilitate review of completed medical surveys by a patient are shown. For example, after submitting the survey response data 142 (shown in FIG. 1), and during a recommendation and treatment process, the patient can access completed medical surveys using the patient interface 110 (also shown in FIG. 1). In the present example, the patient can use the interfaces shown in FIG. 7 to view the survey responses, and to view a recommendation for treatment provided by a medical practitioner that has reviewed the survey responses.

In some implementations, treatment results data that represents a result of applying a recommendation for treatment to a patient may be received. Referring again to FIG. 1, for example, after the medical practitioner has recommended treatment for the patient, and after the patient has undergone the treatment, an evaluation can be performed (e.g., a self-evaluation performed by the patient, a medical evaluation performed by a medical practitioner that specializes in evaluations, or another suitable evaluation), and the evaluation data corresponding to results of the treatment can be provided to the connection platform 102.

In some implementations, treatment results data may be used to update a medical condition data model. For example, the connection platform 102 can update a medical condition data model maintained by the medical condition data source 120 to include received treatment results data. By updating a medical condition data model that provides associations between medical assessment data and recommendations that had been provided for cases with successful outcomes (e.g., positive treatment results), for example, appropriate guidance can be provided to medical practitioners for diagnosing symptoms and/or providing treatment recommendations for patients that experience similar symptoms (e.g., according to provided medical survey responses).

In some implementations, treatment results data may be used to update a medical professional data repository. For example, the connection platform 102 can update the medical practitioner data source 122 to include received treatment results data. By updating medical practitioner data that provides a mapping between various medical symptoms and various medical practitioners to include treatment results data, for example, a process for selecting suitable medical practitioners for diagnosing symptoms and/or providing treatment recommendations can be improved.

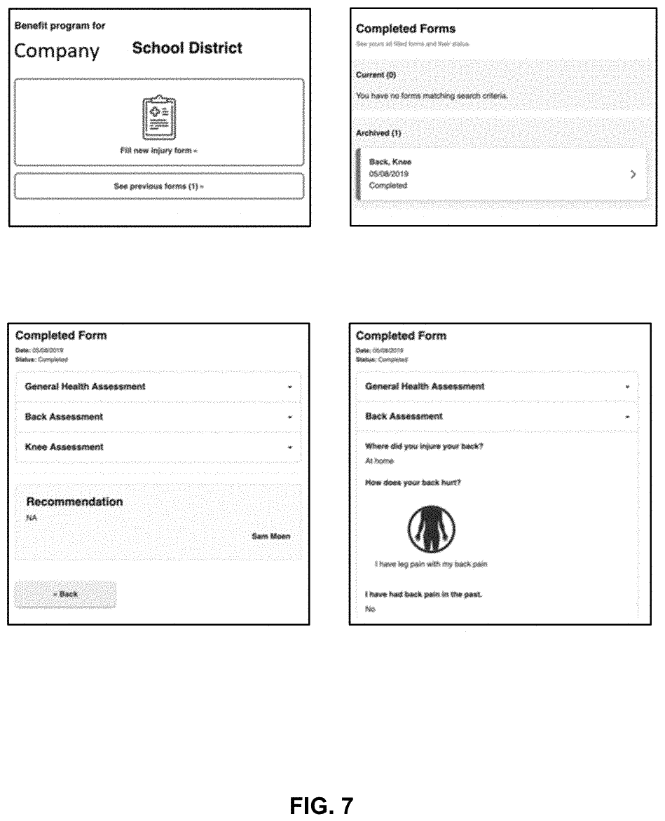

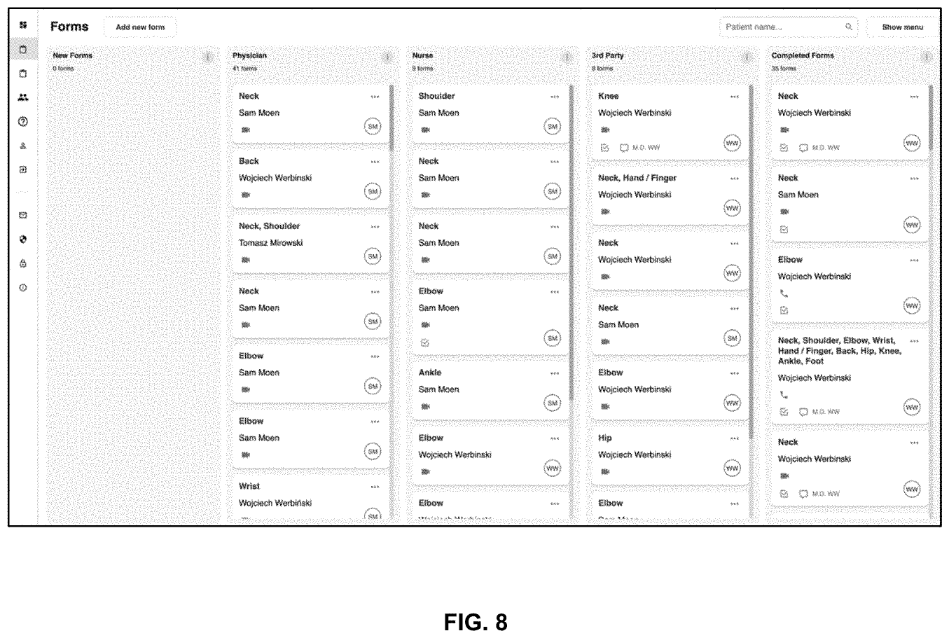

Referring now to FIG. 8, an example user interface that facilitates workflow of a medical practitioner is shown. Using a workflow facilitation interface (e.g., a Kaban board) of the practitioner interface 130 (shown in FIG. 1), for example, a medical practitioner can view their current workflow. For example, new forms can be added through the workflow facilitation interface, and previously generated forms can be located and selected for review.

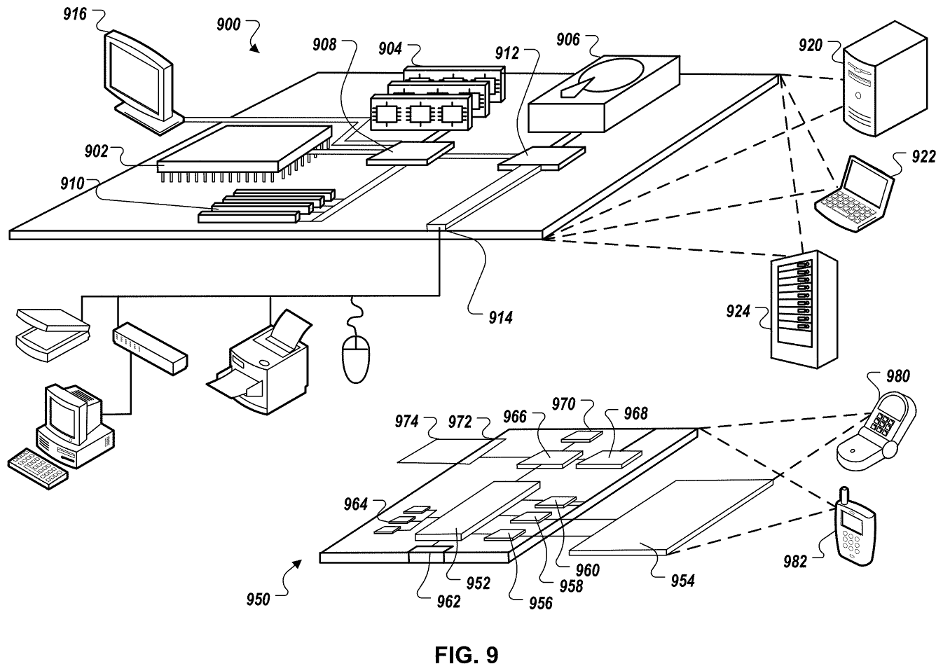

FIG. 9 is a block diagram of computing devices 900, 950 that may be used to implement the systems and methods described in this document, as either a client or as a server or plurality of servers. Computing device 900 is intended to represent various forms of digital computers, such as laptops, desktops, workstations, personal digital assistants, servers, blade servers, mainframes, and other appropriate computers. Computing device 950 is intended to represent various forms of mobile devices, such as personal digital assistants, cellular telephones, smartphones, and other similar computing devices. Additionally, computing device 900 or 950 can include Universal Serial Bus (USB) flash drives. The USB flash drives may store operating systems and other applications. The USB flash drives can include input/output components, such as a wireless transmitter or USB connector that may be inserted into a USB port of another computing device. The components shown here, their connections and relationships, and their functions, are meant to be exemplary only, and are not meant to limit implementations described and/or claimed in this document.

Computing device 900 includes a processor 902, memory 904, a storage device 906, a high-speed interface 908 connecting to memory 904 and high-speed expansion ports 910, and a low speed interface 912 connecting to low speed bus 914 and storage device 906. Each of the components 902, 904, 906, 908, 910, and 912, are interconnected using various busses, and may be mounted on a common motherboard or in other manners as appropriate. The processor 902 can process instructions for execution within the computing device 900, including instructions stored in the memory 904 or on the storage device 906 to display graphical information for a GUI on an external input/output device, such as display 916 coupled to high speed interface 908. In other implementations, multiple processors and/or multiple buses may be used, as appropriate, along with multiple memories and types of memory. Also, multiple computing devices 900 may be connected, with each device providing portions of the necessary operations (e.g., as a server bank, a group of blade servers, or a multi-processor system).

The memory 904 stores information within the computing device 900. In one implementation, the memory 904 is a volatile memory unit or units. In another implementation, the memory 904 is a non-volatile memory unit or units. The memory 904 may also be another form of computer-readable medium, such as a magnetic or optical disk.

The storage device 906 is capable of providing mass storage for the computing device 900. In one implementation, the storage device 906 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 904, the storage device 906, or memory on processor 902.

The high speed controller 908 manages bandwidth-intensive operations for the computing device 900, while the low speed controller 912 manages lower bandwidth-intensive operations. Such allocation of functions is exemplary only. In one implementation, the high-speed controller 908 is coupled to memory 904, display 916 (e.g., through a graphics processor or accelerator), and to high-speed expansion ports 910, which may accept various expansion cards (not shown). In the implementation, low-speed controller 912 is coupled to storage device 906 and low-speed expansion port 914. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 900 may be implemented in a number of different forms, as shown in the figure. For example, it may be implemented as a standard server 920, or multiple times in a group of such servers. It may also be implemented as part of a rack server system 924. In addition, it may be implemented in a personal computer such as a laptop computer 922. Alternatively, components from computing device 900 may be combined with other components in a mobile device (not shown), such as device 950. Each of such devices may contain one or more of computing device 900, 950, and an entire system may be made up of multiple computing devices 900, 950 communicating with each other.

Computing device 950 includes a processor 952, memory 964, an input/output device such as a display 954, a communication interface 966, and a transceiver 968, among other components. The device 950 may also be provided with a storage device, such as a microdrive or other device, to provide additional storage. Each of the components 950, 952, 964, 954, 966, and 968, are interconnected using various buses, and several of the components may be mounted on a common motherboard or in other manners as appropriate.

The processor 952 can execute instructions within the computing device 950, including instructions stored in the memory 964. The processor may be implemented as a chipset of chips that include separate and multiple analog and digital processors. Additionally, the processor may be implemented using any of a number of architectures. For example, the processor 952 may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 950, such as control of user interfaces, applications run by device 950, and wireless communication by device 950.

Processor 952 may communicate with a user through control interface 958 and display interface 956 coupled to a display 954. The display 954 may be, for example, a TFT (Thin-Film-Transistor Liquid Crystal Display) display or an OLED (Organic Light Emitting Diode) display, or other appropriate display technology. The display interface 956 may comprise appropriate circuitry for driving the display 954 to present graphical and other information to a user. The control interface 958 may receive commands from a user and convert them for submission to the processor 952. In addition, an external interface 962 may be provided in communication with processor 952, so as to enable near area communication of device 950 with other devices. External interface 962 may provide, for example, for wired communication in some implementations, or for wireless communication in other implementations, and multiple interfaces may also be used.

The memory 964 stores information within the computing device 950. The memory 964 can be implemented as one or more of a computer-readable medium or media, a volatile memory unit or units, or a non-volatile memory unit or units. Expansion memory 974 may also be provided and connected to device 950 through expansion interface 972, which may include, for example, a SIMM (Single In Line Memory Module) card interface. Such expansion memory 974 may provide extra storage space for device 950, or may also store applications or other information for device 950. Specifically, expansion memory 974 may include instructions to carry out or supplement the processes described above, and may include secure information also. Thus, for example, expansion memory 974 may be provided as a security module for device 950, and may be programmed with instructions that permit secure use of device 950. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 964, expansion memory 974, or memory on processor 952 that may be received, for example, over transceiver 968 or external interface 962.

Device 950 may communicate wirelessly through communication interface 966, which may include digital signal processing circuitry where necessary. Communication interface 966 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 968. In addition, short-range communication may occur, such as using a Bluetooth, WiFi, or other such transceiver (not shown). In addition, GPS (Global Positioning System) receiver module 970 may provide additional navigation- and location-related wireless data to device 950, which may be used as appropriate by applications running on device 950.

Device 950 may also communicate audibly using audio codec 960, which may receive spoken information from a user and convert it to usable digital information. Audio codec 960 may likewise generate audible sound for a user, such as through a speaker, e.g., in a handset of device 950. Such sound may include sound from voice telephone calls, may include recorded sound (e.g., voice messages, music files, etc.) and may also include sound generated by applications operating on device 950.

The computing device 950 may be implemented in a number of different forms, as shown in the figure. For example, it may be implemented as a cellular telephone 980. It may also be implemented as part of a smartphone 982, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the term “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

Although a few implementations have been described in detail above, other modifications are possible. Moreover, other mechanisms for performing the systems and methods described in this document may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

1. A computer system comprising:
a medical condition data model, the medical condition data model providing associations between medical assessment data and areas of concern;
a medical practitioner data repository, the medical practitioner data repository providing a mapping between medical symptoms and medical practitioners; and
one or more computing servers configured to perform operations comprising:
providing medical survey data to a patient computing device, the medical survey data including (i) a symptom question, and (ii) a plurality of medical assessment questions related to a patient response to the symptom question;
receiving, from the patient computing device, the patient response to the symptom question and patient responses to the medical assessment questions;
using the medical condition data model to (i) analyze the received patient responses to the medical assessment questions, and (ii) annotate the medical survey data to indicate one or more medical assessment questions having responses that are areas of concern;
accessing the medical practitioner data repository to select one or more medical practitioners that specialize in recommending treatment for a symptom that corresponds to the patient response to the symptom question;
providing annotated medical survey data to a medical practitioner computing device of at least one of the selected medical practitioners, the annotated medical survey data including the patient responses to the medical assessment questions related to the symptom question, and indications of one or more medical assessment questions having responses that are areas of concern; and
receiving, from the medical practitioner computing device, a recommendation for treatment of a patient associated with the annotated medical survey data.

2. The computer system of claim 1, wherein the plurality of medical assessment questions related to the patient response to the symptom question are presented by the patient computing device after input indicating the patient response to the symptom question is received at the patient computing device.

3. The computer system of claim 2, wherein the patient response to the symptom question includes an indication of one or more symptoms, and the plurality of medical assessment questions include a different set of questions related to each indicated symptom.

4. The computer system of claim 1, wherein the symptom question is presented by the patient computing device as a collection of selectable icons, each selectable icon including a different image that depicts a location and an intensity of a different symptom, the patient response to the symptom question being a selection of one or more of the selectable icons.

5. The computer system of claim 1, the operations further comprising:
providing, to at least two medical practitioner computing devices of at least two of the selected medical practitioners that specialize in recommending treatment for the symptom, a notification that new annotated medical survey data is available for review; and
in response to receiving, from a first one of the at least two medical practitioner computing devices, a confirmation to proceed with review of the new annotated medical survey data:
providing the new annotated medical survey data for presentation by the first one of the at least two medical practitioner computing devices; and
not providing the new annotated medical survey data for presentation by other medical practitioner computing devices.

6. The computer system of claim 1, the operations further comprising:
in response to receiving the recommendation for treatment of the patient:
accessing the medical practitioner data repository to select one or more medical practitioners that specialize in coordinating treatment for the symptom that corresponds to the patient response to the symptom question; and
providing at least some of the medical survey data to a computing device of at least one of the selected medical practitioners that specialize in coordinating treatment of the symptom.

7. The computer system of claim 1, the operations further comprising:
using the medical condition data model to generate an initial recommendation for treatment of the patient; and
wherein providing annotated medical survey data to the medical practitioner computing device includes providing the initial recommendation.

8. The computer system of claim 1, the operations further comprising:
receiving treatment results data that represent a result of applying the recommendation for treatment to the patient.

9. The computer system of claim 8, the operations further comprising:
using the treatment results data to update the medical condition data model.

10. The computer system of claim 8, the operations further comprising:
using the treatment results data to update the medical practitioner data repository; and
wherein accessing the medical practitioner data repository to select one or more medical practitioners includes initially selecting medical practitioners that are associated with past treatment results that are positive overall, and not initially selecting medical practitioners that are associated with past treatment results that are not positive overall.

11. A computer-implemented method comprising:
providing medical survey data to a patient computing device, the medical survey data including (i) a symptom question, and (ii) a plurality of medical assessment questions related to a patient response to the symptom question;
receiving, from the patient computing device, the patient response to the symptom question and patient responses to the medical assessment questions;
using a medical condition data model to (i) analyze the received patient responses to the medical assessment questions, and (ii) annotate the medical survey data to indicate one or more medical assessment questions having responses that are areas of concern;
accessing a medical practitioner data repository to select one or more medical practitioners that specialize in recommending treatment for a symptom that corresponds to the patient response to the symptom question;
providing annotated medical survey data to a medical practitioner computing device of at least one of the selected medical practitioners, the annotated medical survey data including the patient responses to the medical assessment questions related to the symptom question, and indications of one or more medical assessment questions having responses that are areas of concern; and
receiving, from the medical practitioner computing device, a recommendation for treatment of a patient associated with the annotated medical survey data.

12. The computer-implemented method of claim 11, wherein the plurality of medical assessment questions related to the patient response to the symptom question are presented by the patient computing device after input indicating the patient response to the symptom question is received at the patient computing device.

13. The computer-implemented method of claim 12, wherein the patient response to the symptom question includes an indication of one or more symptoms, and the plurality of medical assessment questions include a different set of questions related to each indicated symptom.

14. The computer-implemented method of claim 11, wherein the symptom question is presented by the patient computing device as a collection of selectable icons, each selectable icon including a different image that depicts a location and an intensity of a different symptom, the patient response to the symptom question being a selection of one or more of the selectable icons.

15. The computer-implemented method of claim 11, further comprising:
providing, to at least two medical practitioner computing devices of at least two of the selected medical practitioners that specialize in recommending treatment for the symptom, a notification that new annotated medical survey data is available for review; and
in response to receiving, from a first one of the at least two medical practitioner computing devices, a confirmation to proceed with review of the new annotated medical survey data:
providing the new annotated medical survey data for presentation by the first one of the at least two medical practitioner computing devices; and
not providing the new annotated medical survey data for presentation by other medical practitioner computing devices.

16. The computer-implemented method of claim 11, further comprising:
in response to receiving the recommendation for treatment of the patient:
accessing the medical practitioner data repository to select one or more medical practitioners that specialize in coordinating treatment for the symptom that corresponds to the patient response to the symptom question; and
providing at least some of the medical survey data to a computing device of at least one of the selected medical practitioners that specialize in coordinating treatment of the symptom.

17. The computer-implemented method of claim 11, further comprising:
using the medical condition data model to generate an initial recommendation for treatment of the patient; and
wherein providing annotated medical survey data to the medical practitioner computing device includes providing the initial recommendation.

18. The computer-implemented method of claim 11, further comprising:
receiving treatment results data that represent a result of applying the recommendation for treatment to the patient.

19. The computer-implemented method of claim 18, further comprising:
using the treatment results data to update the medical condition data model.

20. The computer-implemented method of claim 18, further comprising:
using the treatment results data to update the medical practitioner data repository; and
wherein accessing the medical practitioner data repository to select one or more medical practitioners includes initially selecting medical practitioners that are associated with past treatment results that are positive overall, and not initially selecting medical practitioners that are associated with past treatment results that are not positive overall.
